(19) United States
(12) Patent Application Publication
(10) Pub. No.: US 2023/0380892 A1
(43) Pub. Date: Nov. 30, 2023

(54) LEAD AND MEDICAL USE THEREOF

(71) Applicant: Brian H. Spear, Worcester, MA (US)

(72) Inventor: Brian H. Spear, Worcester, MA (US)

(73) Assignee: Prothia s.a.r.l., Paris (FR)

(21) Appl. No.: 17/826,511

(22) Filed: May 27, 2022

(51) Int. Cl. A61B 18/14; A61B 5/287

(52) U.S. Cl. CPC A61B 18/1492; A61B 5/287; A61B 2018/00577

(57) ABSTRACT

A medical lead and method for manufacturing the medical lead where the medical lead includes a lead body having a proximal portion and a flattened distal portion. Additionally, the flattened distal portion includes a resiliently flexible end portion.

### FIELD OF THE INVENTION

This invention generally relates to medical devices and, more particularly, to a medical lead.

### BACKGROUND OF THE INVENTION

Interventional medical devices are commonly used in applications to treat patients diagnosed with various ailments. For example, interventional devices may be utilized to treat various cardiac, neurological, and other conditions with symptoms that include pain, incontinence, insomnia, and movement disorders such as Parkinson's disease and/or epilepsy. Additionally, it is envisioned that interventional devices may be effective in treating a wide range of psychological, emotional, or other physiological conditions.

For example, certain interventional medical devices are designed to deliver mild electrical impulses to neural tissue using an electrical lead. With respect to the treatment of pain, electrical impulses may be directed to specific sites to alleviate the pain. In this case, the neurostimulation may result in effective pain relief and, therefore, in a reduction in use of the medications and/or surgeries used to treat the pain. Other interventional devices are used to deliver high-energy electrical shocks or electrical pulses to the heart in order to treat or cure certain cardiac arrhythmias.

Medical leads are a specific type of interventional device that can be used in the healthcare space for a variety of different medical procedures and applications. In certain applications, the medical lead includes a sensor or active device that is required to remain in relatively constant and stable contact with an internal organ of the body to perform properly the necessary function. Due to the inherent and continuous movement of certain organs within the body due to respiration, cardiac function, etc., it may be difficult for medical leads to maintain constant contact with some internal organs, and in certain cases, precise and stable positioning of the medical lead is clinically critical.

It would therefore be advantageous to have a medical lead designed to maintain the desired contact and position relative to internal organs. Embodiments of the present invention provide such a medical lead. These and other advantages of the invention, as well as additional inventive features, will be apparent from the description of the invention provided herein.

### BRIEF SUMMARY OF THE INVENTION

In one aspect, embodiments of the invention provide a medical lead having a proximal portion and a distal portion. Both portions could be flat such that the medical lead includes an entirely flattened lead body or, in other embodiments, a lead body having a cylindrical proximal portion or a prismatic proximal portion, and a flattened distal portion. The flattened distal portion includes a resiliently flexible end portion.

In a particular embodiment, the resiliently flexible end portion is formed using a resilient metal strip surrounded by a non-metal outer layer. The metal strip may be made from stainless steel, while the non-metal outer layer may include polyurethane or other suitable types of plastic or silicone materials. In alternate embodiments, the resiliently flexible end portion may not have a resilient metal strip, where the resiliency is provided by the material of the flexible end portion rather than by an embedded metal strip.

In certain embodiments, the resiliently flexible end portion includes a first straight section that extends toward the proximal portion, a second straight section that extends toward a tip of the flattened distal portion, and a curved section between the first and second straight sections such that, when deflected from a first position to a second position, the resiliently flexible end portion is biased toward the first position.

The resiliently flexible end portion may further include a second curved or bent section at an end of the second straight section. In a further embodiment, the medical lead includes one or more electrodes attached to a surface of the lead body proximate a tip of the resiliently flexible end portion. Additionally, the lead body may include one or more lumens to accommodate conductive wires from the proximal portion to each of the one or more electrodes. Moreover, in some embodiments, a first electrode of the one or more electrodes is disposed on a first flat side proximate a tip of the resiliently flexible end portion. In yet a further embodiment, a second electrode of the one or more electrodes is disposed on a second flat side of the flattened distal portion opposite the first flat side, and proximate a tip of the resiliently flexible end portion.

One of the one or more of the aforementioned electrodes may be configured for cautery or ablation, for example, or configured to transmit electronic signals for diagnostic purposes. In particular embodiments, the tip of the flattened distal portion has an end face that is angled with respect to a first side of the flattened distal portion, and a first electrode of the one or more electrodes is disposed on the end face. In more particular embodiments, the end face is substantially perpendicular to the first side of the flattened distal portion. In a further embodiment, a second electrode is disposed on the end face. It should be noted that the first and second electrodes may be configured to use high-frequency energy to generate heat between the first and second electrodes.

Embodiments of the medical lead further include a diode disposed on the end face or on the first flat surface, the diode configured to emit or detect light to reveal the amount of oxygen saturation in the blood, for example. Also, the diode may be a light-emitting diode (LED) or a photodiode. In certain embodiments, the end face is rectangular, while in alternate embodiments, the end face is semi-circular and the resiliently flexible end portion is at least partially semi-cylindrical.

In a particular embodiment, the first electrode extends along a portion of the first side, and across the end face. In a more particular embodiment, the first electrode extends along a portion of a second side. The first side may be flat, and the second side may also be flat. The first electrode may be a first screw made from a conductive material, and may also be disposed in a first lumen. Additionally, the first screw may be attached to one or more wires disposed in the first lumen, where the one or more wires are configured to rotate the first screw. In certain embodiments, the medical lead also includes a second screw made from a conductive material, the second screw disposed in a second lumen. The medical lead may further include another lumen configured for an application of negative pressure to promote a sealing engagement between the end face and living tissue.

In a further embodiment, a temperature sensor is disposed on a first flat side proximate a tip of the flattened distal portion. Similarly, a pressure sensor may be disposed on a first flat side proximate a tip of the flattened distal portion. In yet another embodiment, a temperature sensor is disposed on a first flat side proximate a tip of the flattened distal portion, while a pressure sensor is disposed on a second flat side proximate a tip of the flattened distal portion, the second flat side opposite the first flat side.

Embodiments of the medical lead also include a delivery system to guide insertion of the medical lead while inside of a living body. It is envisioned that embodiments of the delivery system could be tubular or shaped like a hollow prism, having a rectangular or triangular cross-section. Furthermore, the proximal portion may include a lumen configured to carry fluids to fill space between the delivery system and the flattened distal portion. In other embodiments, the lead body includes a lumen from the proximal portion to the flattened distal portion, the lumen having an opening disposed on a first flat side proximate a tip of the flattened distal portion, the lumen configured for the application of negative pressure to create suction and promote a sealing engagement between the flattened distal portion and living tissue. The sealing engagement improves the stability of the medical lead, which may be beneficial for certain procedures, such as cardiac ablation, for example. In particular embodiments, the opening includes a ridge along a perimeter of the opening, the ridge configured to enhance the sealing engagement between the flattened distal portion and the living tissue.

In certain embodiments, the lead body includes a lumen from the proximal portion to the flattened distal portion, the lumen having a fiber optic cable to transmit light. In a further embodiment, the proximal portion is cylindrical and gradually transitions to the flattened distal portion, such that a section of the lead body between the cylindrical proximal portion and the flattened distal portion is neither cylindrical nor flat. In a more particular embodiment, the section of the lead body between the cylindrical proximal portion and the flattened distal portion forms a tapered region, and wherein a lumen extends through the cylindrical proximal portion and terminates in an opening in the tapered region. This lumen is useful for irrigating the portion of the medical lead in the delivery system.

One of ordinary skill in the art will recognize that one or more lumens formed in the medical lead may also be used for other functions such as for sampling or infusion. For example, in an intra-uterine application, a clinician may draw amniotic fluid for examination, or fill the cavity with solution to compensate for the loss of fluid. Another example might be delivering fluid to cool non-targeted tissue in the heart during the heat-based ablation of targeted tissue nearby.

In another aspect, embodiments of the invention provide a method of manufacturing the above-described medical lead. The method includes the steps of continuously extruding the lead body to include the proximal portion and the flattened distal portion.

In some embodiments, the method calls for extruding the flattened distal portion with a metal strip and a non-metal outer layer. Other embodiments require extruding the lead body with one or more lumens formed in both the proximal portion and the flattened distal portion. More particular embodiments include placing a conductive wire in one or more of the one or more lumens. Placing the conductive wire may include simultaneously extruding the lead body with the conductive wire in one or more of the one or more lumens. Similarly, some embodiments call for placing a fiber optic cable in one or more of the one or more lumens. Placing the fiber optic cable may include simultaneously extruding the lead body with the fiber optic cable in one or more of the one or more lumens.

Embodiments of the aforementioned method require continuously extruding the lead body with one or more lumens to carry fluids to fill space between a delivery system and the flattened distal portion. Other embodiments call for extruding the lead body with a lumen configured for the application of negative pressure to create suction and promote a sealing engagement between the flattened distal portion and living tissue. Furthermore, the method may include forming an opening in a first flat side proximate a tip of the flattened distal portion, the opening connected to the lumen. More particular embodiments include forming a ridge around a perimeter of the opening, where the ridge is configured to enhance the sealing engagement between the flattened distal portion and living tissue.

In a particular embodiment, the aforementioned method includes continuously extruding the lead body such that the proximal portion is cylindrical or prismatic and gradually transitions to the flattened distal portion, wherein that portion of the lead body between the cylindrical or prismatic proximal portion and the flattened distal portions is neither cylindrical, prismatic, nor flat. More particularly, the method may call for extruding the lead body to include a tapered region, and wherein a lumen extends through the cylindrical or prismatic proximal portion and terminates in an opening in the tapered region.

Embodiments of the aforementioned method also include providing the resiliently flexible end portion with alternately straight and curved sections which are designed to vary the pressure and angles of contact of the tip. Adapting the form and the tissue-contact characteristics is an advantage of the spring-based lead. The method may further include forming an end face on a tip of the flattened distal portion, where the end face is angled with respect to a first side of the flattened distal portion. In a more particular embodiment, the method includes forming an end face that is substantially perpendicular to the first side of the flattened distal portion. Further embodiments of the method call for attaching a first electrode that extends across the end face and along a portion of the first flat side.

The method may further include attaching one or more electrodes to the end face wherein at least one of the one or more electrodes extends across the end face and along a portion of the first side. In some embodiments, the method includes forming a first lumen that extends from the proximal portion to the flattened distal portion, where the first lumen has a first opening in the end face. In other embodiments, the method calls for attaching one or more wires to a first conductive screw and inserting the first conductive screw through the first lumen to the first opening in the end face. The method may include forming a second lumen extending from the proximal portion to the flattened distal portion, such that the second lumen has a second opening in the end face, where the method also includes attaching one or more wires to a second conductive screw and inserting the second conductive screw through the second lumen to the second opening in the end face. Additionally, the method may further include forming a third lumen configured for an application of negative pressure to promote a sealing engagement between the end face and living tissue, thus reducing the risk of migration.

Other aspects, objectives and advantages of the invention will become more apparent from the following detailed description when taken in conjunction with the accompanying drawings.

### BRIEF DESCRIPTION OF THE DRAWINGS

The accompanying drawings incorporated in and forming a part of the specification illustrate several aspects of the present invention and, together with the description, serve to explain the principles of the invention. In the drawings:

FIG. 1A is a schematic representation of a medical lead, constructed in accordance with an embodiment of the invention;

FIGS. 1B and 1C are cross-sectional views for two different locations on the medical lead of FIG. 1A;

FIG. 1D is an enlarged view showing a tip portion of the medical lead of FIG. 1A;

FIGS. 2A-2H and 2J-2N are schematic representations of the resiliently flexible end portion of the medical lead with an arrangement of one or more electrodes, diodes, and/or fiber optic cables attached to a surface of the resiliently flexible end portion, constructed in accordance with embodiments of the invention; and

FIG. 3 is a schematic cross-sectional view showing the medical lead in an application involving use within the heart;

While the invention will be described in connection with certain preferred embodiments, there is no intent to limit it to those embodiments. On the contrary, the intent is to cover all alternatives, modifications and equivalents as included within the spirit and scope of the invention as defined by the appended claims.

### DETAILED DESCRIPTION OF THE INVENTION

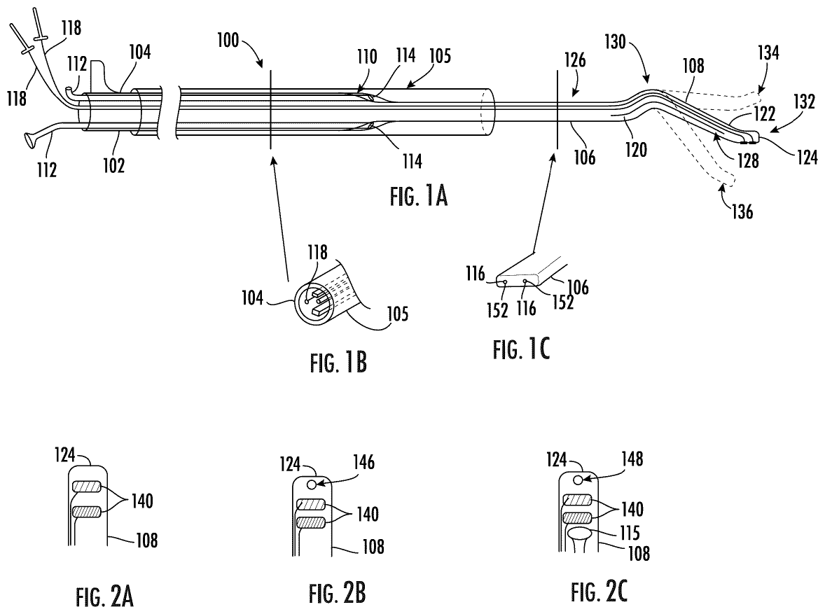

FIG. 1A is a schematic representation of a medical lead 100, constructed in accordance with an embodiment of the invention. FIGS. 1B and 1C are cross-sectional views for two different locations on the medical lead 100 of FIG. 1A. FIG. 1D is an enlarged view showing a tip portion of the medical lead 100 of FIG. 1A. More specifically, FIG. 1B is a schematic illustration showing a cross-section of the proximal portion 104, while FIG. 1C is a schematic illustration showing a cross-section of the flattened distal portion 106.

The medical lead 100 includes a lead body 102 having a proximal portion 104 and a flattened distal portion 106. Furthermore, as shown in FIG. 1A, the flattened distal portion 106 includes a resiliently flexible end portion 108 at an end thereof. The medical lead 100 may also include a tubular delivery system 105 to guide insertion of the medical lead while inside of a living body. As will be explained further below, the proximal portion 104 may be flattened in the same way as the distal portion 106. Additionally, the proximal portion 104 may be cylindrical, prismatic, or some other complex shape to facilitate handling and manipulation by the user.

As can be seen in the embodiment of FIG. 1A, the proximal portion 104 is cylindrical and gradually transitions to the flattened distal portion 106, such that a portion 110 of the lead body 102 between the cylindrical proximal portion 104 and the flattened distal portion 106 is neither cylindrical nor flat. In a more particular embodiment, the portion 110 of the lead body 102 between the cylindrical proximal portion 104 and the flattened distal portion 106 forms a tapered region 110, and wherein one or more fluid-carrying openings or lumens 112 extend through the cylindrical proximal portion and terminates in an opening 114 in the tapered region 110. This aspect of the invention has particular interest in vascular and cardiac applications. Traditional round leads are not adapted to fill the delivery system 105 with fluid to avoid the entrance of blood and risk of coagulation increasing the chances of clotted blood. As designed, this feature does not compromise any of the functions and applications of the flattened distal portion 106 of the lead 100.

In the embodiments shown, the fluid-carrying lumens 112 are configured to carry fluids to fill space between the delivery system 105 and the flattened distal portion 106. This allows for irrigation of the portion of the medical lead 100 that is in the delivery system 105. By keeping the space between the delivery system 105 and the flattened distal portion 106 filled with fluid, blood is prevented from getting into the space and coagulating, which would hinder movement and proper functioning of the lead body 102.

In addition to the fluid-carrying lumens 112 referenced above, the lead body 102 may include one or more lumens 116 formed into the lead body 102 that extend from the proximal portion 104 through to the flattened distal portion 106. The one or more lumens 116 are needed, for example, to accommodate conductive wires 118 from the proximal portion 104 to each of the one or more electrodes that may be provided at or proximate a tip 124 of the resiliently flexible end portion 108.

The lumens 116 make take the form of tubular, typically, though not necessarily, cylindrical openings that extend the length of the medical lead 102. Alternatively, the lumens 116 could include tubular inserts that provide the necessary pathways for the medical lead 100.

Additionally, the lumen 116 may also be used for other functions such as for sampling or infusion. For example, in an intra-uterine application, a clinician may use a particular lumen 116 to draw amniotic fluid for examination, or fill the cavity with solution to compensate for the loss of fluid. Another example involves delivering fluid to cool non-targeted tissue in the heart during the heat-based ablation of targeted tissue nearby. Additionally, wires may be molded or extruded into the lumens 116 during fabrication of the medical lead 100. Alternatively, wires may be inserted into the lumens 116 created during or after extrusion or molding of the medical lead 100.

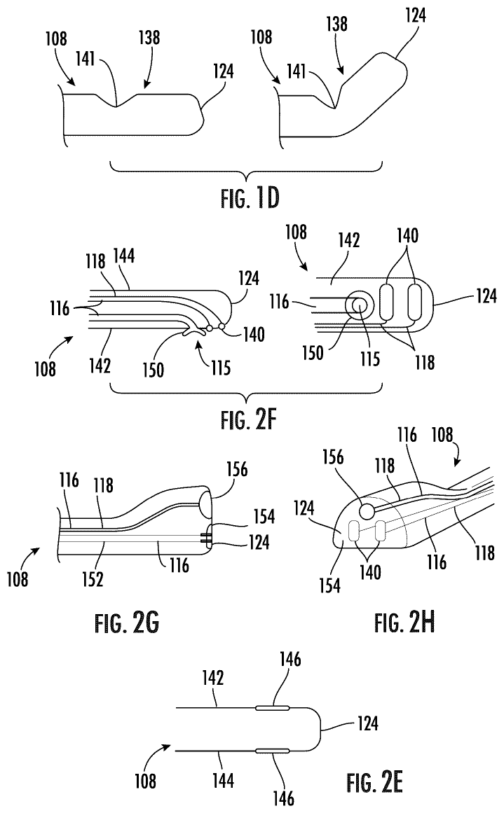

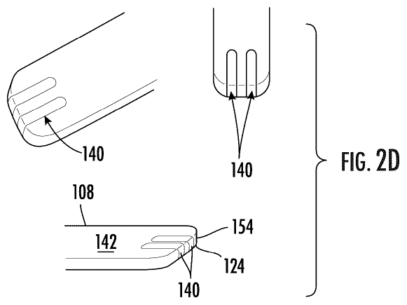

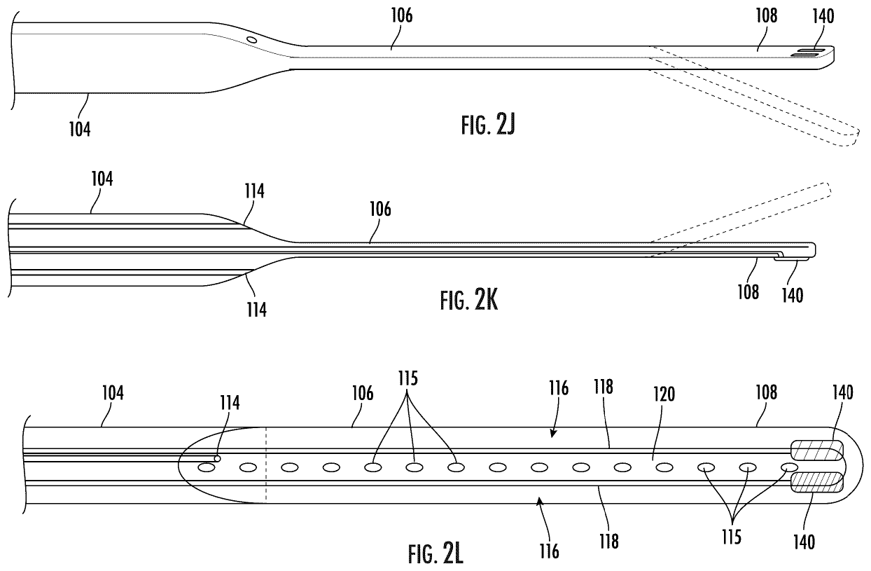

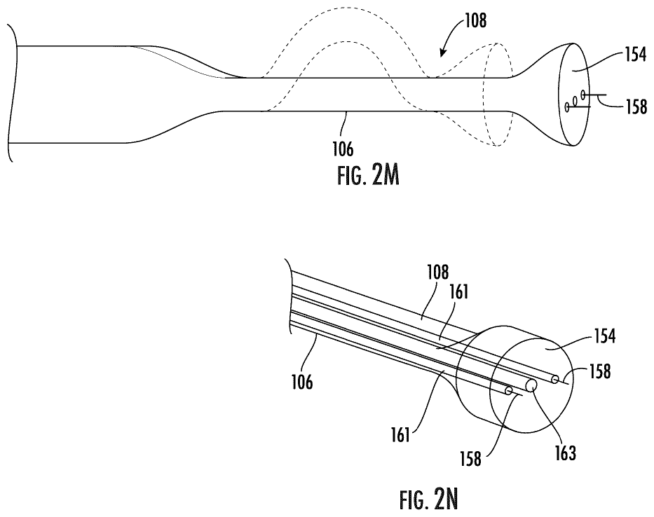

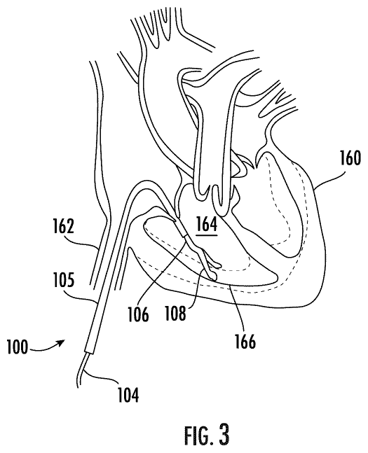

In particular embodiments of the invention, the lead body 102 includes a lumen 116 from the proximal portion 104 to the flattened distal portion 106, in which the lumen 116 terminates at an opening 115 disposed on a first flat side 142 proximate a tip 124 of the flattened distal portion 106, the lumen 116 configured for the application of negative pressure to create suction that promotes a sealing engagement between the resiliently flexible end portion 108 and living tissue. In particular embodiments, the opening 115 includes a ridge 150 (shown in FIG. 2F) along a perimeter of the opening 115. The ridge 150 is configured to enhance the sealing engagement between the flattened distal portion 106 and the living tissue.

In typical embodiments of the invention, the lumens 116 are round. For many applications, a round or circular geometry for the lumen 116 makes sense. However, it may be better to use other geometries when extruding the lumen 116, for example, rectangular, triangular, or other shapes, depending on the particular application.

In certain embodiments such as illustrated in the cross-sectional view of FIG. 1C. In FIG. 1C, the lead body 102 includes two lumens 116 extending from the proximal portion 104 to the flattened distal portion 106. In this example, the lumens 116 accommodate a fiber optic cable 152 to transmit light through the lead body 102. In another example, the two lumens 116 might typically carry conductive wires 118 to the one or more electrodes attached proximate a tip of the resiliently flexible end portion 108.

In a further embodiment, the resiliently flexible end portion 108 includes a first straight section 126 the extends toward the proximal portion 104, a second straight section 128 that extends toward the tip 124 of the flattened distal portion 106, and a curved section 130 between the first and second straight sections 126, 128 such that, when deflected from a first position 132 to a second position 134 or third position 136 (see broken line positions of resiliently flexible end portion 108 in FIG. 1A), the resiliently flexible end portion 108 is biased back toward the first position 132.

FIG. 1D shows enlarged cross-sectional image of the tip 124 of the resiliently flexible end portion 108. As shown in this drawing, the resiliently flexible end portion 108 may further include a second curved or bent section 138 at an end of the second straight section. The second curved or bent section 138 may include a groove 141 on one of the flat surfaces of the end portion 108, where the groove 141 facilitates the bending of the resiliently flexible end portion 108. The bending gives the tip 124 more flexibility and enables the tip 124 to have better, more consistent contact with living tissue.

In particular embodiments of the invention, the resiliently flexible end portion 108 is formed using a resilient metal strip 120 surrounded by a non-metal outer layer 122. The resilient metal strip 120 may be made from stainless steel, while the non-metal outer layer 122 may include silicone, polyurethane, or other suitable types of plastic materials. The metal strip 120 is formed and shaped or bent as necessary in order to function like a flat spring and provide the aforementioned resiliency. It should be understood that there are embodiments of the medical lead 100 in which the resiliently flexible end portion 108 is formed without using a resilient metal strip. A non-metal resilient strip may be used or none at all, where the resiliency is provided by the material (metallic or non-metallic) of the flexible end portion 108.

FIGS. 2A-2G are schematic representations of the resiliently flexible end portion 108 each with an arrangement of one or more electrodes 140, diodes, and/or fiber optic cables attached to a surface of the resiliently flexible end portion 108 proximate the tip 124, in accordance with embodiments of the invention. Each of the resiliently flexible end portions 108 has a first flat surface 142 and a second flat surface 144 (shown in FIGS. 2E and 2F) opposite the first flat surface 142. FIG. 2A shows an embodiment with two electrodes 140 positioned adjacent to each other on the first flat surface 142.

FIG. 2B shows an embodiment with two electrodes 140 positioned adjacent to each other on the first flat surface 142 and a pressure sensor 146 located on the first flat surface 142 between electrodes 140 and the tip 124. FIG. 2C shows an embodiment with two electrodes 140 positioned adjacent to each other on the first flat surface 142 and a temperature sensor 148 located on the first flat surface 142 between electrodes 140 and the tip 124. FIG. 2C also includes the above-described opening 115 disposed on the first flat side 142 to promote a sealing engagement, when negative pressure is applied, between the resiliently flexible end portion 108 and living tissue.

In particular embodiments, the tip 124 of the resiliently flexible end portion 108 has an end face 154 that is substantially perpendicular to a first flat side 142 of the flattened distal portion 106. FIG. 2D shows an embodiment of the resiliently flexible end portion 108 with two electrodes 140 that extend across the end face 154, and along a portion of the first flat side 142. In one example, the aforementioned electrodes 140 are configured for cautery. The arrangement of FIG. 2D allows the tip 124 and the flat first face 142 to perform the necessary cutting or cauterization. In a more particular embodiment, the electrodes also extend along a portion of a second flat side 144.

FIG. 2E shows an embodiment of the resiliently flexible end portion 108 with a pressure sensor 146 on the flat first side 142 to sense blood pressure, for example, and a pressure sensor 146 on the flat second side 144 to sense contact pressure with living tissue, for example. Thus, the flat profile of the resiliently flexible end portion 108 allows for measurements to be taken independently from both flat sides 142, 144 of the end portion 108. In alternate embodiments, one or both of the pressure sensors 146 can be replaced by other types of sensors, e.g., temperature sensor 148. Additionally, the embodiment of FIG. 2E may include one or more electrodes 140 placed on both flat sides 142, 144 of the end portion 108.

FIG. 2F shows a side view of an embodiment of the resiliently flexible end portion 108 arranged similarly to the embodiment of FIG. 2C with two electrodes 140 at the end of two lumens 116 with conductive wires 118 connected to the two electrodes 140. The two electrodes 140 are positioned adjacent to each other on the first flat surface 142, and the above-described opening 115 disposed on the first flat side 142. In FIG. 2F, the opening 115 at the end of the lumen 116 includes a ridge 150 along a perimeter of the opening 115. The ridge 150 is configured to enhance the sealing engagement between the flattened distal portion 106 and the living tissue. As arranged, when negative pressure is applied, the ridge 150 improves the suction holding effect of the resiliently flexible end portion 108 near the tip 124.

FIGS. 2G and 2H show plan and perspective views, respectively, with embodiments of the resiliently flexible end portion 108 in which the end face 154 is not rectangular, as in the above-described embodiments. Instead, the end face 154 in these embodiments is semi-circular and the resiliently flexible end portion 108 is at least partially semi-cylindrical. Other shapes are possible for the resiliently flexible end portion 108. In an alternate embodiment, the end face 154 and resiliently flexible end portion 108 could be triangular. FIGS. 2G and 2H each include a diode 156 disposed on the end face 154 at the end of lumen 116 with a conductive wire 118 connected to the diode 156. The diode 156 may be a light-emitting diode (LED) configured to emit, or a photodiode configured to detect light.

In alternate embodiments, the diode could be attached to the first or second flat surface 142, 144 as well as the end face 154. FIG. 2G also includes a fiber optic cable 152 disposed in lumen 116 and extending to the end face 154. The fiber optic cable 152 transmits light to or from the end face 154. FIG. 2H includes the diode 156 and two electrodes 140 attached to the end face 154 at the end of two lumens 116 with conductive wires 118 connected to the two electrodes 140.

In the embodiment of FIG. 2H, the two electrodes 140 attached to the end face 154 may be configured to use high-frequency energy to generate heat between the two electrodes 140, allowing the tip 124 of the resiliently flexible end portion 108 to be used as a surgical cutting tool. This application using two electrodes 140 and high-frequency energy to generate heat between the two electrodes 140 may be performed using an end portion 108 that does not include the diode 156.

FIGS. 2J, 2K, and 2L are plan views of alternate embodiments of the flattened distal portion 106 and resiliently flexible end portion 108. The embodiment includes with two electrodes 140 at the end of two lumens 116 with conductive wires 118 connected to the two electrodes 140. For the sake of convenience, the flattened distal portion 106 is shown as relatively short in comparison to a more typical embodiment of the medical lead 100. In the embodiment shown, the flattened distal portion 106 and the resiliently flexible end portion 108 are straight with no bends or curves. However, the end portion 108 is highly flexible and resilient, as shown in the broken line segments in FIGS. 2J and 2K. It should also be noted that FIG. 2L provides a transparent view in which the resilient metal strip 120 is visible inside the flattened distal portion 106. As shown, the resilient metal strip 120 includes multiple openings 115 placed sequentially along the length of the metal strip 120. The openings 115 facilitate bonding with the material of the surrounding outer layer which flows through the openings 115 and joins with the resilient metal strip 120. As explained above, the material of the surrounding outer layer could be made from silicone, polyurethane, or some other suitable plastic material.

FIGS. 2M and 2N show perspective views of another embodiment of the resiliently flexible end portion 108 of the flattened distal portion 106. Similar to FIGS. 2G and 2H, the embodiment of FIGS. 2M and 2N has a flat end face 154 that is substantially perpendicular to the sides of the flattened distal portion 106 and resiliently flexible end portion 108. But rather than semi-circular, the end face 154 of FIGS. 2M and 2N is circular and has two conductive screws 158 (i.e., screws made from a conductive material such as metal) protruding from respective lumens 160. In the embodiment shown, a third lumen 163 is positioned between the two lumens 160. The third lumen 163 is configured for the application of negative pressure to promote a sealing engagement between the flat end face 154 and living tissue so that the screws can be threaded into the tissue to create a much stronger attachment for the medical lead 100.

Once the end face 154 is secured to the living tissue at the desired location using negative pressure applied via the third lumen 163, the conductive screws 158 can be inserted by the user into the proximal portion 104 and maneuvered through the lumens 160 to the resiliently flexible end portion 108 and out of the flat end face 154. The conductive screws 158 could be attached to the end of a wire or group of wires that provide torque control to rotate the screws clockwise or counterclockwise at the flat end face 154. Once inserted into the living tissue, the conductive screws 158 act as electrodes and can be used for ablation, cautery, or to transmit signals for diagnostic purposes, e.g., signals for an ECG. As shown in FIG. 2M, the flexible end portion 108 may be configured to deform and bend, for example, when the end face 154 comes into hard contact with living tissue. By deforming and absorbing the force of the contact, the flexible end portion 108 can prevent any damage to the living tissue.

The medical lead 100 and the various embodiments described above may be manufactured by continuously extruding the lead body 102 to include the above-described proximal portion 104 and the flattened distal portion 106. The proximal portion 104 may be extruded as flat, cylindrical, or prismatic. The extrusion method may include simultaneously extruding the flattened distal portion 106 with the resilient metal strip 120 and a non-metal outer layer 122. Furthermore, the method may include extruding the lead body 102 with one or more lumens 116 formed in both the proximal portion 104 and the flattened distal portion 106. More particular embodiments include placing a conductive wire 118 in one or more of the one or more lumens 116. Placing the conductive wire 118 may include simultaneously extruding the lead body 102 with the conductive wire 118 in one or more of the one or more lumens 116. Similarly, some embodiments call for placing a fiber optic cable 152 in one or more of the one or more lumens 116. Placing the fiber optic cable 152 may include simultaneously extruding the lead body 102 with the fiber optic 152 cable in one or more of the one or more lumens 116.

Embodiments of the aforementioned method require extruding the lead body 102 with one or more fluid-carrying lumens 112 to carry fluids to fill space between the delivery system 105 and the flattened distal portion 106. Other embodiments call for extruding the lead body 102 with the lumen 116 configured for the application of negative pressure to promote a sealing engagement between the flattened distal portion 106 and living tissue. Furthermore, the method may include the step of forming an opening 115 in a first flat side 142 proximate a tip of the flattened distal portion, the opening 115 connected to the lumen 116. More particular embodiments of the method include forming a ridge 150 around a perimeter of the opening, 115 where the ridge 150 is configured to enhance the sealing engagement between the flattened distal portion 106 and living tissue.

In a particular embodiment of the aforementioned method of manufacturing, the method calls for continuously extruding the lead body 102 such that the proximal portion 104 is cylindrical or prismatic and gradually transitions to the flattened distal portion 106, wherein that portion of the lead body 102 between the cylindrical or prismatic proximal portion 104 and the flattened distal portion 106 is neither cylindrical, prismatic, nor flat. More particularly, the method may call for extruding the lead body 102 such that the portion of the lead body 102 between the cylindrical or prismatic proximal portion 104, and the flattened distal portion 106 is a tapered region 110, and where a fluid-carrying lumen 112 extends through the cylindrical or prismatic proximal portion and terminates in an opening in the tapered region 110.

Embodiments of the aforementioned method also include providing the resiliently flexible end portion with alternately straight 126, 128 and curved 130 sections such that, when deflected from a first position 132 to a second position 134 or a third position 136, the resiliently flexible end portion 108 is biased back toward the first position 132. The method may further include forming an end face 154 on a tip 124 of the flattened distal portion 106, where the end face 154 is substantially perpendicular to a first flat side 142 of the flattened distal portion 106. Further embodiments of the method call for attaching a one or more electrodes 140 that extends across the end face 154 and along a portion of the first flat side 142. Further embodiments could include those in which the one or more electrodes 140 further extend along a portion of the second flat side 144.

FIG. 3 is a schematic cross-sectional view showing the medical lead 100 in a specific application involving use of the lead 100 within the heart 160. In this particular application, the medical lead 100 is used to track the movement of the ventricular wall 166 of the heart 160 as it is beating. As shown in FIG. 3, the delivery system 105 guides the flat proximal portion 104 through a major vein 162 into the right ventricle 164 of the heart 160. The flattened distal portion 106 and the resiliently flexible end portion 108 are shown inside the right ventricle 164 with the resiliently flexible end portion 108 positioned against the ventricular wall 166.

To effectively track the movement of the ventricular wall 166 of the heart 160 as it is beating, the contact between the resiliently flexible end portion 108 and the ventricular wall 166 must be as continuous and stable as possible. The resilience and flexibility of the end portion 108 allows this portion of the medical lead 100 to move and flex independently as the heart 160 contracts so that the entire medical lead 100 does not have to move along with the end portion 108. Additionally, as explained above, the resiliently flexible end portion 108 may include one or more openings 115 connected to the lumen 116, where the opening 115 is configured to enhance sealing engagement between the flattened distal portion 106 and the ventricular wall 166.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A medical lead comprising:
a lead body having a proximal portion and a flattened distal portion, wherein the flattened distal portion includes a resiliently flexible end portion.

2. The medical lead of claim 1, wherein the resiliently flexible end portion is formed using a resilient metal strip surrounded by a non-metal outer layer.

3. The medical lead of claim 2, wherein the metal strip is made from stainless steel.

4. The medical lead of claim 2, wherein the non-metal outer layer includes silicone or polyurethane.

5. The medical lead of claim 1, wherein the resiliently flexible end portion includes a first straight section that extends toward the proximal portion, a second straight section that extends toward a tip of the flattened distal portion, and a curved section between the first and second straight sections such that, when deflected from a first position to a second position, the resiliently flexible end portion is biased toward the first position.

6. The medical lead of claim 1, wherein the resiliently flexible end portion includes a second curved or bent section at an end of the second straight section.

7. The medical lead of claim 1, further comprising one or more electrodes attached to a surface of the lead body proximate a tip of the resiliently flexible end portion.

8. The medical lead of claim 7, wherein the lead body includes one or more lumens to accommodate conductive wires from the proximal portion to each of the one or more electrodes.

9. The medical lead of claim 7, wherein a first electrode of the one or more electrodes is disposed on a first flat side proximate a tip of the resiliently flexible end portion.

10. The medical lead of claim 9, wherein a second electrode of the one or more electrodes is disposed on a second flat side of the flattened distal portion opposite the first flat side, and proximate a tip of the resiliently flexible end portion.

11. The medical lead of claim 7, wherein one of the one or more electrodes is configured for cautery or ablation.

12. The medical lead of claim 7, wherein one of the one or more electrodes is configured to transmit signals for diagnostic purposes.

13. The medical lead of claim 7, wherein the tip of the flattened distal portion has an end face that is angled with respect to a first side of the flattened distal portion, and wherein a first electrode of the one or more electrodes is disposed on the end face.

14. The medical lead of claim 13, further comprising a second electrode disposed on the end face.

15. The medical lead of claim 13, wherein the first and second electrodes are configured to use high-frequency energy to generate heat between the first and second electrodes.

16. The medical lead of claim 13, further comprising a diode disposed on the end face or on the first side, the diode configured to emit or detect light.

17. The medical lead of claim 16, wherein the diode is a light-emitting diode (LED) or a photodiode.

18. The medical lead of claim 13, wherein the end face is rectangular.

19. The medical lead of claim 13, wherein the end face is semi-circular and the resiliently flexible end portion is at least partially semi-cylindrical.

20. The medical lead of claim 13, wherein the first electrode extends along a portion of the first side, and across the end face.

21. The medical lead of claim 20, wherein the first electrode extends along a portion of a second side.

22. The medical lead of claim 20, wherein the first side is flat, and the second side is flat.

23. The medical lead of claim 13, wherein the end face is substantially perpendicular to the first side of the flattened distal portion.

24. The medical lead of claim 13, wherein the first electrode is a first screw made from conductive material, the first screw disposed in a first lumen.

25. The medical lead of claim 24, wherein the first screw is attached to one or more wires disposed in the first lumen, the one or more wires configured to rotate the first screw.

26. The medical lead of claim 24, further comprising a second electrode which is a second screw made from conductive material, the second screw disposed in a second lumen.

27. The medical lead of claim 24, further comprising another lumen configured for an application of negative pressure to promote a sealing engagement between the end face and living tissue.

28. The medical lead of claim 1, wherein a temperature sensor is disposed on a first flat side proximate a tip of the flattened distal portion.

29. The medical lead of claim 1, wherein a pressure sensor is disposed on a first flat side proximate a tip of the flattened distal portion.

30. The medical lead of claim 1, wherein a temperature sensor is disposed on a first flat side proximate a tip of the flattened distal portion, and a pressure sensor is disposed on a second flat side proximate a tip of the flattened distal portion, the second flat side opposite the first flat side.

31. The medical lead of claim 1, further comprising a delivery system to guide insertion of the medical lead while inside of a living body.

32. The medical lead of claim 31, wherein the proximal portion includes a lumen configured to carry fluids to fill space between the delivery system and the flattened distal portion.

33. The medical lead of claim 31, wherein the delivery system is tubular or shaped like a hollow prism.

34. The medical lead of claim 1, wherein the lead body includes a lumen from the proximal portion to the flattened distal portion, the lumen having an opening disposed on a first flat side proximate a tip of the flattened distal portion, the lumen configured for an application of negative pressure to promote a sealing engagement between the flattened distal portion and living tissue.

35. The medical lead of claim 34, wherein the opening includes a ridge along a perimeter of the opening, the ridge configured to enhance the sealing engagement between the flattened distal portion and the living tissue.

36. The medical lead of claim 1, wherein the lead body includes a lumen from the proximal portion to the flattened distal portion, the lumen having a fiber optic cable to transmit light.

37. The medical lead of claim 1, wherein the proximal portion is cylindrical and gradually transitions to the flattened distal portion, such that a section of the lead body between the cylindrical proximal portion and the flattened distal portion is neither cylindrical nor flat.

38. The medical lead of claim 37, wherein the section of the lead body between the cylindrical proximal portion and flattened distal portion forms a tapered region, and wherein a lumen extends through the cylindrical proximal portion and terminates in an opening in the tapered region.

39. The medical lead of claim 1, wherein the proximal portion is flat.

40. The medical lead of claim 1, wherein the proximal portion is cylindrical or prismatic.

41. A method of manufacturing the medical lead of claim 1, comprising the steps of:
continuously extruding the lead body to include the proximal portion and the flattened distal portion.

42. The method of claim 41, wherein continuously extruding the lead body comprises extruding the flattened distal portion with a metal strip and a non-metal outer layer.

43. The method of claim 41, wherein continuously extruding the lead body comprises extruding the lead body with one or more lumens formed in both the proximal portion and the flattened distal portion.

44. The method of claim 43, further comprising placing a conductive wire in one or more of the one or more lumens.

45. The method of claim 44, wherein placing the conductive wire comprises simultaneously extruding the lead body with the conductive wire in one or more of the one or more lumens.

46. The method of claim 43, further comprising placing a fiber optic cable in one or more of the one or more lumens.

47. The method of claim 46, wherein placing the fiber optic cable comprises simultaneously extruding the lead body with the fiber optic cable in one or more of the one or more lumens.

48. The method of claim 41, wherein continuously extruding the lead body comprises extruding the lead body with one or more lumens to carry fluids to fill space between a delivery system and the flattened distal portion.

49. The method of claim 41, wherein continuously extruding the lead body comprises extruding the lead body with a lumen configured for an application of negative pressure to promote a sealing engagement between the flattened distal portion and living tissue.

50. The method of claim 49, further comprising forming an opening in a first flat side proximate a tip of the flattened distal portion, the opening connected to the lumen.

51. The method of claim 49, further comprising forming a ridge around a perimeter of the opening, the ridge configured to enhance the sealing engagement between the flattened distal portion and living tissue.

52. The method of claim 41, wherein continuously extruding the lead body comprises extruding the lead body such that the proximal portion is cylindrical and gradually transitions to the flattened distal portion, wherein that section of the lead body between the cylindrical proximal portion and flattened distal portions is neither cylindrical nor flat.

53. The method of claim 52, wherein extruding the lead body such that the cylindrical proximal portion gradually transitions to the flattened distal portion comprises extruding the lead body to include the tapered region, and wherein a lumen extends through the cylindrical proximal portion and terminates in an opening in the tapered region.

54. The method of claim 41, further comprising providing the resiliently flexible end portion with alternately straight and curved sections such that, when deflected from a first position to a second position, the resiliently flexible end portion is biased toward the first position.

55. The method of claim 41, further comprising forming an end face on a tip of the flattened distal portion, the end face being angled with respect to a first side of the flattened distal portion.

56. The method of claim 55, further comprising attaching one or more electrodes to the end face wherein at least one of the one or more electrodes extends across the end face and along a portion of the first side.

57. The method of claim 55, wherein forming the end face comprised forming the end face substantially perpendicular to the first side of the flattened distal portion.

58. The method of claim 55, further comprising forming a first lumen extending from the proximal portion to the flattened distal portion, the first lumen having a first opening in the end face.

59. The method of claim 58, further comprising attaching one or more wires to a first conductive screw and inserting the first conductive screw through the first lumen to the first opening in the end face.

60. The method of claim 58, further comprising forming a second lumen extending from the proximal portion to the flattened distal portion, the second lumen having a second opening in the end face, attaching one or more wires to a second conductive screw and inserting the second conductive screw through the second lumen to the second opening in the end face.

61. The method of claim 60, further comprising forming a third lumen configured for an application of negative pressure to promote a sealing engagement between the end face and living tissue.
